(19) United States
(12) Patent Application Publication
(10) Pub. No.: US 2005/0260585 A1
(43) Pub. Date: Nov. 24, 2005

(54) Poison/antidote genetic systems for the selection of genetically modified eucaryote cells or organisms

(76) Inventors: Cedric Szpirer, Fleurus (BE); Michel Milinkovitch, Jemeppe-Sur-Sambre (BE); Philippe Gabant, Brussels (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/507,923

(22) PCT Filed: Mar. 19, 2003
(86) PCT No.: PCT/BE03/00045

§ 371 Date: Jul. 19, 2005

Related U.S. Application Data
(60) Provisional application No. 60/365,938, filed on Mar. 19, 2002.

(51) Int. Cl. C12Q 1/68; C12N 5/04

(52) U.S. Cl. 435/6; 435/325; 435/419

(57) ABSTRACT

The present invention is related to a recombinant eucaryote cell or organism having incorporated in its genome a genetic construct made of at least one nucleotide sequence encoding a toxic gene (TOX) under the control of an inducible promoter/operator genetic sequence and possibly a selectable marker. The present invention is also related to a production and selection method-of-genetically modified eucaryote cells or organisms having integrated into their genome foreigner (exogenous) DNA fragment(s) by using said recombinant eucaryote cells or organisms.

### FIELD OF THE INVENTION

The present invention is related to poison/antidote genetic systems for the selection of genetically modified eucaryote cells (plant, yeast, and animal cells or plant, yeast, and animal organisms).

### BACKGROUND OF THE INVENTION AND STATE OF THE ART

When attempting to produce transgenic organism (plant, animal or yeast), one is necessarily faced with the major problem of assessing the actual integration of an exogenous DNA fragment into the genome of said organism or in some or all of its cells.

For example in a transgenic plant, Agrobacterium tumefaciens is commonly used as the mean by which a DNA fragment can be introduced in a plant cell genome (cf. document JP2001029092 and publication of Zambryski et al. 1988).

Unfortunately, when the exogenous DNA fragment or gene is expressed, it is not always possible for the experimenter to assess of a stable insertion of said DNA fragment in the plant cell genome.

Indeed, among the several copies of the DNA fragment that are effectively targeted by Agrobacterium to the nucleus of the plant cell, most are transitorily expressed, and only a tiny fraction (between 1/1000 to 1/10000) are stably integrated into the genome (publication of Y. Chupeau, Médecine/science 2001, vol. 17, p. 856-866).

Furthermore, the exact location of the exogenous DNA integrated in the organism genome is basically unpredictable (Tinland B, Trends Plant Science 1996, vol. 1, p. 178-184, Bechtold et al. Genetics 2000, vol. 155, p. 1875-1887).

Furthermore, the rate of homologous recombination into plant cells seems to be about one hundred time less frequent that the rate of “illegitimate” recombination (Chupeau, Médecine/science 2001, vol. 17, p. 856-866 and Kempin et al. Nature 1997, vol. 389, p. 802-803).

### AIMS OF THE PRESENT INVENTION

The present invention aims to provide method and means for the characterization and the selection of genetically modified cells and pluricellular organisms that have correctly integrated foreigner exogenous DNA fragment(s) into their genome, preferably cells and organisms which have integrated at a specific location said foreigner (exogenous) DNA fragment.

A further aim of the present invention is to allow the selection of said cells and organisms obtained by rare homologous recombination events.

### SUMMARY OF THE INVENTION

The present invention is based upon a method and a poison/antidote genetic system used for the selection of stable insertion of foreigner (exogenous) DNA fragment(s) into the genome of an eucaryote cell or a pluricellular organism but which allows also the precise targeting of said insertion in a specific (preferably predefined) location in said genome and the possibility to easily characterize the presence, the integrity and the correct orientation of said inserted foreigner (exogenous) DNA fragment(s) into the genome of said cell or organism.

The present invention is based upon a genetic construct which comprises a toxic gene, preferably a poison, under the control of an inducible promoter/operator genetic sequence and a selectable marker (such as an antibiotic resistance gene), said genetic construct being introduced into an eucaryote cell or eucaryote organism.

The genetic construct of system according to the invention comprises a genetic sequence encoding a toxic molecule (TOX) (preferably a nucleotide sequence encoding a poison protein) under the control of an inducible promoter/operator genetic sequence and possibly a selectable marker (such as an antibiotic resistance gene A). Said genetic construct is introduced into the eucaryotic cell or organism so as to produce a recombinant cell or organism.

Said introduction, is preferably obtained by the use of known transfection or viral infection means which allow the introduction of said genetic construct and its expression into an eucaryote genome.

An eucaryote genome means DNA sequences which are present in the nucleus of the eucaryotic cell or in specific cell compartments (chloroplasts and mitochondria) which comprises also genetic materials.

Preferred means suitable for the introduction of said genetic construct into the genome of a plant cell are for example a modified Ti plasmid corresponding to a Ti plasmid containing said genetic construct flanked by LB and RB repeats genetic borders (Hellens et al., 2000, Plant Mol Biol 42 vol 6, p. 819-832; Dennis et al., WO0018939).

Said modified plasmid will be as follows
LB-TOX-selectable marker A-RB

Transfection of plant cells could be obtained by an infection of plant cells with the strain Agrobacterium tumefaciens containing this modified Ti plasmid. A nuclease (VirD) will excise the LB-TOX-A-RB fragment which is then targeted to the nucleus of the plant cells (via the action of VirD2 and ViE2 proteins)(Rossi et al., 1996, Procede Natl Acad Sci USA 93 vol 1, p. 126-130). The plant cells that have integrated said construct, i.e. the recombinant plant cells, are selected by using the marker A. The marker of the insertion is determined by sequencing or screening the genomic library with a DNA probe corresponding to the toxic gene (or to the marker A) or using any other molecular biology techniques (genetic amplification like PCR, mapping, etc) well known by the person skilled in the art.

Each recombinant cell line obtained through this protocol can be thereafter used for a precise targeted integration of any foreigner (exogenous) DNA fragment(s) into the genome of the cell by homologous recombination into said genetic construct or system.

The exogenous DNA fragment is preferably carried by a nucleic construct and the selection of genetically modified cells having integrated correctly said exogenous DNA fragment will be achieved through the expression of the toxic gene.

Indeed, the genetic construct according to the invention, which carries the toxic gene and is integrated in the genome of the recombinant cell, as well as the nucleic construct which carries the exogenous DNA fragment, are so constructed that homologous recombination between said constructs may occur. Under these conditions, only the cells which have integrated the exogenous DNA fragment through homologous recombination will survive, because they have replaced the construct according to the invention containing the toxic gene by the exogenous DNA fragment.

If someone wants to further insure that not only the toxic gene, but also the marker A is removed during the recombination event, the marker A can be bordered by two toxic genes (different or the same) and the construct will be as follows:
LB-TOX-selectable marker A-TOX-RB.

Hence, any cell with a recombination event removing two toxic genes would necessarily lack the selectable marker A as well.

The toxic gene present in the genetic construct according to the invention could be a member of a bacterial poison/antidote family or derivative thereof (genetically modified sequence(s) of said poison/antidote selected by the person skilled in the art, in order to improve their poisonous characteristics). Said poisonous molecules are for instance genes coding for the CcdB, ParE, RelE, Kid, Doc, MazE, PemK, HoK proteins (Engelberg-kulka and Glaser, 1999n Annu Rev Microbiol. 53, p. 43-70; Gabant et al., 2002, In Recent Res Devel Plasmid Biol p. 15-28). Previously, it was shown that some of them are active in Eucaryote cells (yeast Saccharomyces cerevisiae and human cells, Kristoffersen et al., 2000, Appl. Environ. Microbiol. 66, p 5524-5526; Yamamoto et al., 2002, FEBS letters 519, p 191-194.) This activity was described for use in controlling the survivability of cells when these cells are released in the environment (“gene containment”) (WO99/58652, Gerdes et al.).

The antidotes are for example the genes coding for the CcdA, Kis, Phd, PemI, SoK proteins.

The risk of a possible “leaking” of the expression of said poisonous gene (low, but non-zero activity of inducible promoter) is resolved through the use of an antidote gene under the control of an inducible promoter (said inducible promoter being the same as the inducible promoter controlling the expression of the poisonous gene mentioned here above, or being different from it). Under this scheme, the antidote gene is added to the construct according to the invention in order to control the expression or activity of the poisonous protein and will have the following configuration LB-ANTITOX-TOX-selectable marker A-RB.

Another possibility is the introduction of said antidote genetic sequence in an episomal DNA introduced also in the eucaryote cell or eucaryotic organism.

Therefore, the poison/antidote genetic systems or construct may consist of two elements, a stable toxin and an unstable antidote (RNA or protein sequence). These antidotes (peptides) could be degraded by a specific ATP-dependent protease (such as the Lon protease of Escherichia coli which degrades the CcdA antidote of the ccd system, Van Melderen et al., 1994, Mol Microbiol 11 vol 6, p. 1151-1157).

Preferably, the gene encoding this protease that is specific of the antidote degradation is introduced in a transgenic eucaryotic cell or organism in order to allow a rapid effective activity of the poison upon its target.

Although the present invention is suitable for the integration of exogenous DNA fragment into a plant cell, this system could be also applied to insertion of foreigner (exogenous) DNA construct in any type of eucaryote cell or pluricellular organism (yeast cell, animal cells or organisms, such as mammalian cell or insect cell) preferably with the proviso that said cell or organism is not a human germs cell line, a human zygote, a human embryo, or a human individual.

Furthermore, the combination of a toxic gene with an inducible promoter in a plant cell opens the possibility to use the toxic gene as an efficient and entirely transgenic-line specific herbicide.

This application of the present invention requires the production of a transgenic line of a plant species or breed with a specific genetic construct.

Said improved genetic construct is made of a gene encoding a toxic molecule, preferably a gene encoding a poison/protein which is under the control of a promoter/operator genetic sequence inducible by a non-toxic natural or artificial compound.

A non-toxic natural or artificial compound means a compound which is or is not toxic for the plant or for the environment.

In the present case, the obtained transgenic plants would not need to be eradicated, because the promoter/operator genetic sequence can be activated in order to allow the expression of the genes encoding the toxic molecule by the addition of the above-mentioned compound.

For example, specific promoter/operator genetic sequences are those controlled by the addition of chemical compounds (Zuo and Chua, 2000 Curr Opin Biotechnol 11 vol 2, p. 146-151; Zuo et al., 2000, Plant Journal 24 vol 2, p. 265-273).

In addition, the promoter/operator genetic sequence could be also tissue specific in order to allow that some specific portion of plant cells or tissues should be genetically modified (leaves, flowers, etc).

Furthermore, the promoter/operator genetic sequence could be activated or repressed by a compound that is synthesized by the plant or plant cell itself preferably at a specific stage of its development or in a specific tissue.

Therefore, the tissue specific or development-stage specific compound could be a compound encoded by a gene that is artificially inserted into the plant genome.

In some specific cases, it could be obtained a genetic construct which comprises a nucleus sequence encoding a specific toxic molecule (poison protein) fused to sequence guiding the fusion protein to the poison target.

For example, if the toxic molecule is the CcdB poison protein, said sequence can be fused to a signal protein targeting the construct product to the nucleus where the CcdB target (gyrase) is located and active.

Furthermore, some specific applications will require the use of a specific poison sequence whose activity against an eucaryote cell is suboptimal.

Therefore, the present invention could be improved by the introduction (within or separately from the exogenous above-mentioned genetic construct) of a sequence encoding the poison target. Said introduction and modification could be a modification of the cell genome of a procaryote cell, an eucaryote cell or an eucaryote organism.

For example, the recombinant cell including a recombinant procaryote cell or organism could be modified by the introduction into its genome of the target sequence of said toxic molecule preferably a bacterial gyrase gene, if the CcdB poison is used (and if said CcdB poison is not efficient enough upon the corresponding eucaryotic gyrase).

The co-occurrence of the bacterial and eucaryotic gyrases will not be problematic as the CcdB+prokaryotic gyrase complex will exhibit a dominant effect as in prokaryotes.

Furthermore, the target poison can also be guided to specific cell compartments (chloroplasts, mitochondria) where the poison aims to be also active.

Therefore, the genetic construct according to the invention could be also integrated directly into said specific cell compartments (chloroplasts, mitochondria) or the cell may comprise also one or more specific cell compartments (chloroplasts, mitochondria) wherein the antidote genetic sequence to said toxic molecule are also integrated as episomal DNA sequence.

1-14. (canceled)

15. A recombinant eucaryote cell or organism with the proviso that it is not an element selected from the group consisting of a human germ cell line, a human zygote, a human embryo and a human individual, said cell or organism having incorporated in its genome
i) a genetic construct made of at least one nucleotide sequence and optionally a selectable marker, said sequence encoding a toxic gene (TOX) under the control of an inducible promoter/operator genetic sequence; and
ii) a genetic sequence (ANTITOX) encoding an antidote molecule to said toxic molecule with the condition that the sequence encoding the antidote molecule is not present natively in said cell or organism, and wherein the genetic sequence encoding the antidote is added to the construct or is in an episomal DNA introduced in the eucaryote cell or organism.

16. The recombinant eucaryote cell or organism according to claim 15, wherein the genetic sequence encoding the antidote molecule is under the control of an inducible promoter/operator genetic sequence.

17. The recombinant eucaryote cell or organism according to claim 15, wherein the genetic sequence encoding the toxic molecule is a genetic sequence encoding a poison protein, selected from a poison/antidote group.

18. The recombinant eucaryote cell or organism according to claim 17, wherein the genetic sequence encoding the toxic molecule is a genetic sequence encoding a poison protein selected from the group consisting of CcdB, ParE, RelE, Kid, Doc, MazF and Hok proteins.

19. The recombinant eucaryote cell or organism according to claim 15, wherein said cell or organism is a plant cell or a plant.

20. The recombinant eucaryote cell or organism according to claim 15, wherein said cell or organism is an animal cell or an animal.

21. The recombinant eucaryote cell or organism according to claim 20, wherein said animal cell or animal is a mammalian cell or a mammal.

22. The recombinant eucaryote cell according to claim 15, wherein said cell is a yeast cell.

23. The recombinant eucaryote cell or organism according to claim 15, wherein the inducible promoter/operator genetic sequence is induced by a non-toxic compound.

24. The recombinant eucaryote cell or organism of claim 23, wherein the non-toxic compound is an exogenous compound or a compound that is synthesized by the eucaryote cell or organism itself at a specific stage of its development or in a specific tissue.

25. The recombinant eucaryote cell or organism according to claim 15 further comprising a genetic sequence integrated into its genome, wherein said genetic sequence is the target of the toxic molecule or said genetic sequence encodes the target of the toxic molecule.

26. The recombinant eucaryote cell or organism according to claim 15, wherein the genetic construct is integrated into the genome of a specific cell compartment.

27. The recombinant eucaryote cell or organism according to claim 26, wherein the specific cell compartment is a chloroplast or a mitochondrion.

28. The recombinant eucaryote cell or organism according to the claim 15, wherein the selectable marker is bordered by two different or identical toxic genes.

29. A production and selection method of a genetically modified eucaryote cell or organism having integrated into its genome an exogenous DNA fragment, said method comprising the steps of (i) providing a recombinant eucaryote cell or organism with a genetic construct carrying a toxic gene integrated therein; (ii) providing a construct carrying an exogenous DNA fragment; (iii) obtaining integration of said exogenous DNA fragment in the genome of the recombinant eucaryote cell at the insertion site where the genetic construct is integrated; (iv) selecting the genetically modified eucaryote cell or organism having integrated said exogenous DNA fragment under conditions allowing the expression of the toxic molecule in said cells or organisms; and (v) recovering said genetically modified eucaryote cells or organisms which do not express said toxic molecule following the integration of the exogenous DNA fragment.

30. The production and selection method according to claim 29, wherein said exogenous DNA fragment is integrated into the genome of the recombinant eucaryote cell or organism by homologous recombination between the sequence of said exogenous DNA fragment and the sequence of the genetic construct integrated into the genome of the recombinant eucaryote cell or organism.

31. The production and selection method according to claim 29, wherein said eucaryote cell or organism is a plant cell or a plant transfected by a Ti-plasmid incorporating the toxic gene.

32. The method of claim 31, wherein a complete transgenic plant is obtained from the recovered genetically modified plant cell.

33. The production and selection method of claim 31, wherein the Ti-plasmid incorporating the toxic gene is present in Agrobacterium tumefaciens.
